(19) United States
(12) Patent Application Publication
(10) Pub. No.: US 2005/0150503 A1
(43) Pub. Date: Jul. 14, 2005

(54) Tummy lift

(76) Inventor: Thomas W. Votel, St. Paul, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/031,053

(22) Filed: Jan. 7, 2005

Related U.S. Application Data
(60) Provisional application No. 60/535,587, filed on Jan. 9, 2004.

(51) Int. Cl. A61F 5/37

(52) U.S. Cl. 128/846

(57) ABSTRACT

An apparatus and method for repositioning the abdominal fat tissue of a patient by a single operator to enable medical personnel to gain access to certain areas of a patient's body, for example, to provide medical care, without risk of injury to the operator and/or health care worker, and with minimal discomfort to the patient.

### FIELD OF THE INVENTION

The invention relates to an apparatus and method for assisting in repositioning the fat tissue of a patient to enable medical personnel to gain access to certain areas of a patient's body to provide care to the affected area. The invention more particularly relates to an apparatus which gives a single health care worker the ability to reposition a patient's abdominal fat tissue and provide medical care without risk of injury to the health care worker or discomfort to the patient.

### BACKGROUND OF THE INVENTION

Certain medical procedures require that health care workers have access to areas of a patient's body that may, in some cases, be partially or completely covered by an abdominal panniculus, or “apron,” of fat tissue. This apron of fat tissue may make it difficult for health care workers to provide needed medical care to these patients. Certain procedures, such as inserting catheters, changing dressings, inserting suppositories, etc., may be made much more difficult by the presence of this apron of fat.

This situation is arising more frequently in recent years, particularly in the United States, where an estimated 15% of the population is now classified as morbidly obese. Many of these individuals accumulate a large mass of fat tissue in the lower abdominal area producing a panniculus (apron of fat) that hangs dependently over the groin, genital area and upper thighs.

In order to move this apron from the field of work, two nurses typically use a sheet as a sling to pull the apron up and away from the affected area while a third health care worker provides care. This can be uncomfortable for the patient and puts two nurses at risk of injury. Furthermore, the sheet may not provide enough friction to hold the apron out of the way, and may also not conform to the apron of fat.

What is needed is a device or system that allows a single health care worker to reposition a morbidly obese patient's apron of fat to give the health care worker the ability to provide needed care, while minimizing the risk of injury and/or discomfort to both the health care worker and the patient.

### BRIEF SUMMARY OF THE INVENTION

Certain embodiments of the invention include a system for displacing abdominal fat tissue of a patent that uses an adjustable sling that can be attached to existing support members, such as the side rails of a hospital bed, to support and move the fat tissue.

Certain embodiments of the invention include an apparatus for repositioning a patient's abdominal fat tissue in which a sling is coupled to support members, such as a portion of a hospital bed, with end portions extending from either side of the sling. The apparatus is configured to reposition the fat tissue by adjusting the length of one or both end portions and/or moving the support members.

Certain embodiments of the invention include a method of repositioning a patient's abdominal fat tissue using the system and apparatus described herein.

### BRIEF DESCRIPTION OF THE DRAWINGS

FIG. 1 is a perspective view of an apparatus for assisting in repositioning the fat tissue of a patient in accordance with an embodiment of the invention.

FIG. 2 is a perspective view of an attaching means for operatively coupling the apparatus of FIG. 1 to a support member in accordance with an embodiment of the invention.

FIG. 3 is a perspective view of the apparatus of FIG. 1 operatively coupled to side rails on either side of a hospital bed in accordance with an embodiment of the invention.

FIG. 4 is a perspective view of the apparatus of FIG. 1 adapted for use with a patient in a hospital bed in accordance with an embodiment of the invention.

FIG. 5(a)-5(c) are side views of the apparatus of FIG. 1 in different stages of use with a patient in accordance with an embodiment of the invention.

### DETAILED DESCRIPTION

The following discussion is presented to enable a person skilled in the art to make and use the invention. Various modifications to the illustrated embodiments will be readily apparent to those skilled in the art, and the generic principles herein may be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the invention. Skilled artisans will recognize the examples provided herein have many useful alternatives which fall within the scope of the invention.

Embodiments of the invention are not limited to medical applications, and may be employed in various situations which require the repositioning of a person's fat tissue. However, for purposes of illustration only, the invention is described below in the context of providing medical care to a patient. It is to be further understood that the present invention is not limited to repositioning abdominal fat tissue and may be utilized for other purposes. For purposes of illustration only, however, the invention is described below in the context of abdominal fat tissue.

A person having a body mass index (or “BMI,” a ratio between a person's height and weight) of 40 or above (e.g., someone who is 5-foot-4 and about 233 pounds) is considered “morbidly obese,” indicating that the risk of illness or disease related to that person's obesity increases dramatically at or above this degree of obesity. The percentage of the U.S. population now classified as morbidly obese is estimated to be about 15%, and has increased significantly in recent years. Many morbidly obese individuals accumulate a large mass of abdominal fat tissue in the lower abdominal area producing an abdominal panniculus, or “apron” of fat that hangs dependently over the groin or genital area, and may reach the upper thighs. This apron may make it difficult for medical personnel to provide care to this area of some patients. For example, inserting catheters, changing dressings, inserting suppositories, etc. may be made more difficult by the presence of such an apron on a patient.

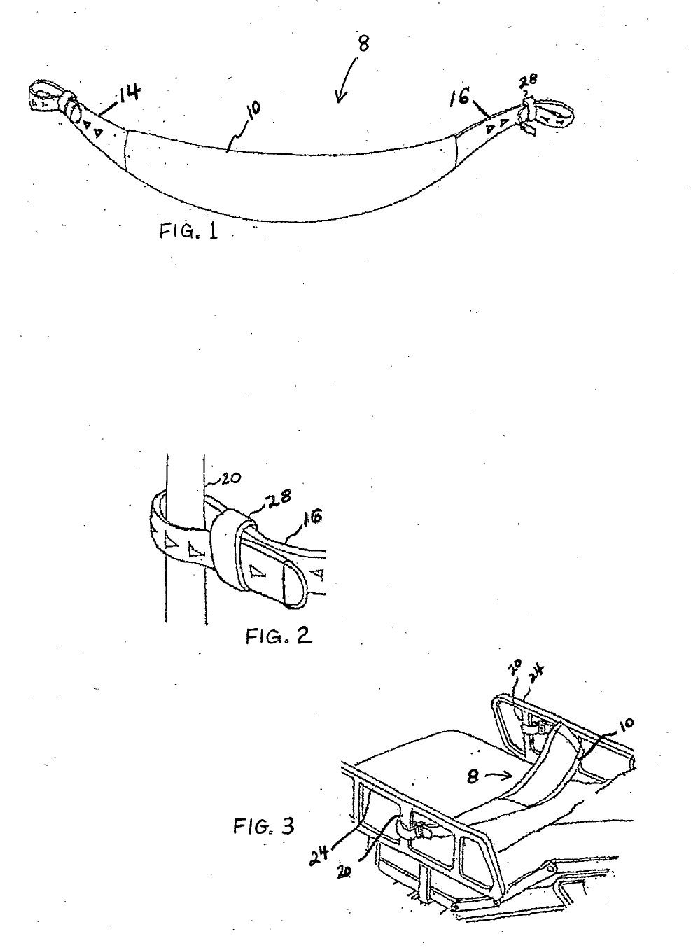

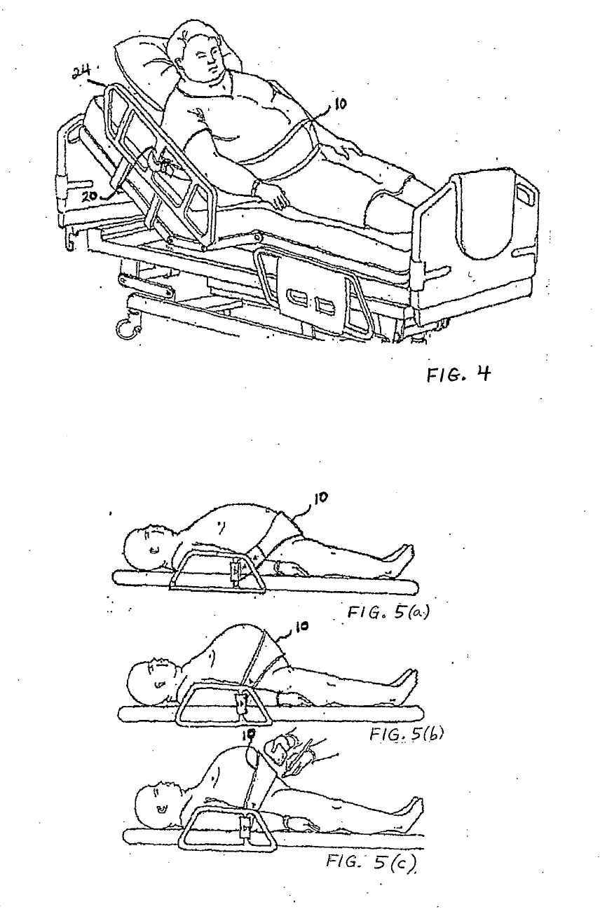

FIG. 1 is a perspective view of apparatus 8 for repositioning an apron of abdominal fat tissue of a morbidly obese patient in accordance with an embodiment of the invention. As shown in FIG. 1, the apparatus comprises a sling portion 10 and two end portions 14, 16 disposed on two sides of the sling portion 10. The distal ends of the end portions 14, 16 are adapted to be operatively coupled to a support member 20 (shown in FIG. 2). In some embodiments, the distal end of one or both end portions 14, 16 may be adapted for adjustable attachment to a support member, such as by use of adjustable D-ring type fastener 28, for example, as shown in FIG. 1. FIG. 4 is a perspective view of apparatus 8 illustrating one possible embodiment as it may be used with patient 30.

The sling portion 10 may be made using suitable materials adapted to support a patient's apron of fat. The sling portion 10 may be made of a stretchable or non-stretchable material, according to various embodiments of the invention. For patient comfort, a stretchable material may be desirable to conform to the shape of the apron, for example, to assist in supporting the patient's apron of fat. However, a relatively non-stretchable material may be preferable in some circumstances, for example, when supporting a relatively large or heavy apron of fat tissue.

If a stretchable material is desired for use in forming the sling portion 10, a suitable material, such as Spandex®, may be utilized. If a non-stretchable material is desired, any suitable material may be utilized. In one possible embodiment using a relatively non-stretchable material, 100% cotton twill, 6.25 oz./yard weight, 2-ply construction may be used to form the sling portion 10. The 2-ply construction gives the sling portion 10 added strength and is able to withstand repeated hospital laundering.

In another embodiment, the sling portion 10 may have a material laminated thereto to allow heat and moisture to be transferred away from the body of the patient. Such a suitable fabric may be a fibrous material, such as Coolmax™ fabric manufactured by DuPont. The fabric is simply laminated to the sling portion 10 such that the fabric is adjacent the body of the patient when the apparatus 8 is used. Alternately, instead of being laminated to the sling portion 10, the fabric (Coolmax™) may be used as the material forming the sling portion 10.

In one embodiment, the sling portion 10 and the end portions 14, 16 may be made of a continuous piece of material. For example, the material that forms the sling portion 10 may be formed so that end portions 14, 16 are simply extensions of the material that makes up sling portion 10. In such an embodiment, it may be preferable to shape the apparatus 8 so that the sling portion 10 is wider than the end portions 14, 16, in order to provide adequate support to the apron of fat while facilitating operative coupling of end portions 14, 16 to a suitable support member 20 (see FIG. 2).

In an alternate embodiment, one or both of the end portions 14, 16 may be constructed independently of the sling portion 10, and operably connected to the sling portion 10 at one end thereof. The end portions 14, 16 in such an embodiment may be made of a different material than the sling portion 10, for example, and operably connected to the sling portion 10 by any suitable means, such as by stitching, rivets, hook and loop fasteners, etc. The hook material and loop fabric, if used, may be of any type well-known in the industry which would form a hook and loop type fastener, such as Velcro brand, and may be secured to the sling portion 10 and end portion 14 or 16 using a suitable method, such as by stitching.

In embodiments where the end portions 14, 16 are constructed independently of the sling portion 10, the end portions 14, 16 may comprise straps as are known in the art. The straps may be made of any suitable material, such as a cotton or canvas web material, for example. The straps may preferably comprise a cotton webbing, approximately 1 and 3/4 inches wide.

As shown in FIG. 2, end portions 14, 16, or straps, are adapted to be operatively coupled to a support member 20 by any suitable attaching means. Support member 20 may comprise a structure that is relatively stable, such as an upright bar that may be part of an upper portion of a bed rail 24 (FIG. 3), for example. The stability of support member 20 provides resistance to support the apron of fat with the sling portion 10. At least one of the straps, for example end portion 16 in FIG. 2, comprises an adjustable attaching means, such as D-ring type fastener 28, that permits adjusting the length of the strap. The strap 16 may be shortened by an operator by pulling the distal end of the strap 16, which places the sling portion 10 under tension and tends to lift the apron of fat up toward the patient's head and out of the way for improved access to affected areas of a patient's body. (See FIG. 5(c)).

The adjustable attaching means used near the distal end of at least one of the straps may be an adjustable D-ring type fastener 28, as described above, or may comprise other suitable adjustable attaching means that permit altering the length of one of the end portions 14, 16 from a first position to a second position, thereby tightening or loosening the apparatus with respect to the apron of fat. The adjustable attaching means is preferably operable by a single operator, and may comprise buckle-type fasteners, ratcheting fasteners, and hook-and-loop fasteners such as Velcro brand hook-and-loop fasteners, as well as other types of adjustable fasteners as are known in the art. In an embodiment in which a buckle-type adjustable fastener is used, the distal end of an end portion 14 or 16 may have a loop 28 through which end portion 14 or 16 may pass and be supported thereby.

In one embodiment, the end portions 14, 16 are preferably operatively connected to support members 20 at locations that are nearer the patient's head than is the sling portion 10 when in supportive engagement with the apron of fat. This configuration responds to an increase in tension across apparatus 8 and/or sling 10 by tending to pull the apron of fat generally up and toward the head of the patient.

The end portions 14, 16 may be operatively coupled to a support member 20 as described above. As shown in FIG. 3, support member 20 may comprise an upright bar that is a portion of upper bed rail 24, according to an embodiment of the invention. However, support member 20 may comprise any suitably stable structure, including, but not limited to the headboard 32, the upper and lower horizontal bars of the upper bed rail 24, the frame portion of the bed, and a portion of the patient's body, such as the shoulder and neck area, for example.

FIGS. 5(a)-5(c) show side views of apparatus 8 in different stages of use with a patient, in accordance with an embodiment of the invention. FIG. 5(a), for example, shows apparatus 8 as it may be initially configured for use with patient 30. As shown, the end portions 14, 16 (only end portion 14 can be seen in FIG. 5(a)) are operatively coupled to support members 20 on either side of patient 30. Support member 20 can be a suitably stable fixture, such as a portion of the hospital bed; FIG. 5(a) shows an upright bar of upper bed rail 24 being used as support member 20, although the invention is not so limited. The location of the operative coupling of end portions 14, 16 is preferably nearer the head of patient 30 than is the sling portion 10 of apparatus 8 when initially configuring apparatus 8 for use, as shown in FIG. 5(a).

FIG. 5(b) shows that the abdominal fat of patient 30 may be repositioned by shortening the overall length of apparatus 8, according to one embodiment of the invention. The length of apparatus 8 may be shortened, for example, by adjusting the length of one or both end portions 14, 16 to be a shorter length using adjustable attaching means, including but not limited to adjustable D-ring fasteners, buckle-type fasteners, ratcheting fasteners, and hook-and-look fasteners.

FIG. 5(c) shows a health care worker 40 providing medical care to the areas of the patient's body previously obstructed by the apron of fat.

In one alternate embodiment, the support members 20 may be moved toward the patient's head (with or without tightening end portions 14, 16). The bed rail 24 could slide toward the head of the bed manually and be locked into place, for example, or the bed rail 24 could be motorized and operated slowly to move bed rail 24 longitudinally in a direction toward the head of patient 30. A clamp on top of the bed rail 24 could also be used as support member 20 according to an embodiment.

In another embodiment of the invention, the apparatus 8 could be positioned on the patient while the bed is partially reclined, as shown in FIG. 4. Then, when the patient's upper torso is lowered by flattening the bed, the apparatus 8 will pull the apron of abdominal fat upward and out of the way of areas previously obstructed by the apron.

Mechanical means may be employed to move support members 20 (i.e., bed rail 24) longitudinally. Mechanical means may include a sliding rail arrangement, pulleys, ratchets, etc., to move and hold the support members in place and thereby reposition the apron of fat. Motorized means may also be incorporated to facilitate the movement of support members 20. Motorized means may utilize worm gears to facilitate the movement of the support members 20 (i.e., side rails) longitudinally with respect to the patient's body. Worm gears may provide a slower, more controlled application of tension to the apparatus 8, thereby reducing the chance of causing injury or discomfort to the patient 30.

In a preferred embodiment of the invention, a single health care worker may be able to both reposition the patient's apron of fat using the apparatus 8, and also provide medical care to areas of the patient previously obstructed by the apron, without causing risk of injury to themselves or the patient 30.

Thus, embodiments of a TUMMY LIFT APPARATUS AND METHOD are disclosed. One skilled in the art will appreciate that the present invention can be practiced with embodiments other than those disclosed. The disclosed embodiments are presented for purposes of illustration and not limitation, and the present invention is limited only by the claims that follow.

1. A system for displacing abdominal fat tissue of a patient, comprising a sling portion with first and second end portions, an attaching means disposed on each of the first and second end portions adapted to attach each of the first and second end portions to support members disposed on either side of a patient, one of the attaching means allowing one of the end portions to be adjusted from a first length to a second length, thereby displacing the fat tissue.

2. An apparatus for repositioning a patient's abdominal fat tissue comprising:
a sling portion having first and second end portions, each end portion adapted to be operatively coupled to a first and second support member, respectively,
the first and second support members being disposed laterally on either side of the patient, and
the sling portion adapted to reposition and support the patient's abdominal fat tissue.

3. The apparatus of claim 2 wherein the sling portion is adapted to be placed laterally across a patient's abdomen such that increasing tension on the sling portion tends to move the patient's abdominal fat tissue toward a head of the patient.

4. The apparatus of claim 3 wherein a distal end of both end portions is adapted to be operatively coupled to the respective support member at a location nearer the head of the patient than the sling portion.

5. The apparatus of claim 3 wherein one of the first and second end portions is adjustably coupled to the respective support member to adjust the amount of tension on the sling portion.

6. The apparatus of claim 3 wherein one of the first and second support members is adapted to move to adjust the amount of tension on the sling portion.

7. The apparatus of claim 6 wherein one of the first and second support members is adapted to move longitudinally with respect to the patient to adjust the amount of tension on the sling portion.

8. The apparatus of claim 6 wherein one of the first and second support members is adapted to rotate so that of distal end of one of the first and second end portions winds around the support member and shortens the length of the end portion to adjust the amount of tension on the sling portion.

9. The apparatus of claim 2 wherein the sling portion comprises a stretchable material.

10. The apparatus of claim 2 wherein the sling portion comprises a material that transfers heat and moisture away from a patient.

11. The apparatus of claim 2 wherein one of the first and second end portions comprises a strap operatively coupled to the sling portion.

12. The apparatus of claim 11 wherein the strap is adapted to be operatively connected to a support member by adjustable fastener means.

13. The apparatus of claim 12 wherein the adjustable fastener means comprises a D-ring fastener.

14. The apparatus of claim 12 wherein the adjustable fastener means comprises a hook and loop fastener arrangement.

15. The apparatus of claim 12 wherein the adjustable fastener means comprises a quick release fastener.

16. The apparatus of claim 12 wherein the adjustable fastener means comprises a buckle type fastener.

17. The apparatus of claim 2 wherein one of the first and second support members comprises a portion of a hospital bed.

18. The apparatus of claim 17 wherein the support member further comprises a side rail of a hospital bed.

19. The apparatus of claim 17 wherein the support member further comprises a clamp located on a portion of a side rail of a hospital bed.

20. A method of repositioning a patient's abdominal fat tissue to facilitate access to an area of medical treatment, the method comprising:
providing an apparatus in supportive engagement with a patient's abdominal fat tissue, the apparatus having a sling portion having first and second end portions, each end portion adapted to be operatively coupled to a portion of a hospital bed disposed laterally on either side of the patient;
operatively coupling the first and second end portions to a portion of a hospital bed disposed laterally on either side of the patient.

21. The method of claim 20 further comprising:
adjusting the length of one of the end portions to adjust the position of the patient's abdominal fat tissue, wherein the one of the end portions is adjustably coupled to the portion of a hospital bed disposed laterally on either side of the patient.

22. The method of claim 21 wherein the step of adjusting the length of one of the end portions comprises pulling a distal end of the end portion.

23. The method of claim 20 further comprising:
moving the portion of the hospital bed longitudinally by mechanical means to adjust the position of the patient's abdominal fat tissue.
